(19) United States
(12) Patent Application Publication
(10) Pub. No.: US 2013/0066259 A1
(43) Pub. Date: Mar. 14, 2013

(54) Colon Irrigation Device and Method

(71) Applicant: Delmar Vogel, North Vancouver (CA)

(72) Inventor: Delmar Vogel, North Vancouver (CA)

(21) Appl. No.: 13/228,209

(22) Filed: Sep. 8, 2011

(51) Int. Cl. A61M 3/02

(52) U.S. Cl. 604/28; 604/31; 604/39

(57) ABSTRACT

The present invention provides a highly effective device and method for performing human colon irrigation. It comprises a bypass line having a non-return valve connected to a warm water supply line where in the non-return valve operates in proportion to back pressure as it develops in the colon thereby allowing gentler filling of colon and limiting pressure in the colon to a maximum safe level. Benefits include patient comfort and a safeguard against damaging the intestine. The present invention also provides a novel housing for a colon irrigation device which provides easy access to the internal part of the device without the need for disconnecting water or electrical supply lines.

### STATEMENT REGARDING FEDERALLY FUNDED RESEARCH

This invention was not made by any agency of the United States Government nor under any contract with any agency of the United States Government.

### TECHNICAL FIELD OF THE INVENTION

The invention relates to medical equipments and more particularly to colon irrigation devices and a method for performing colon irrigation.

### BACKGROUND OF THE INVENTION

The colon may retain large amounts of matters, which accumulate and do not move out of the colon during long periods of time or less mobile matter due to bowel deficiencies. Such accumulation of more or less permanent residence of feces material in the colon may lead to toxic effects, inflammation of the lining of the colon, or other serious complications. To avoid such difficulties, colon irrigation is suggested. Colon irrigation is also known by various names such as colon lavaging, colon cleansing, colon washing, and colon hydrotherapy, which are herein after referred to as colon irrigation.

Numerous types of systems and devices for irrigating and cleansing the colon can be found in the prior art. The prior art systems and devices apparatus typically include a fluid (such as water) inlet for introducing water into the apparatus, means for purifying the water such as a filter system, means for controlling flow and pressure of the fluid, and a fluid outlet which is in turn connected to a rectal speculum via a tubing. The prior art systems and apparatus for colon irrigation/hydrotherapy include temperature and flow components, which are controlled by electronic components, all of which are contained generally within the same housing or compartment.

Traditionally, colonic machines are used by therapists in the following manner. A speculum that is secured to flexible hosing is inserted into the anal canal by the attendant and a supply of warm water injected via the speculum into the colon. While the therapist massages the patient's abdomen he observes the pressure of the water within the speculum and patient from a gauge. As the water fills the colon pressure increases. Once the pressure reaches a selected upper level, such as one pound per square inch, above which the patient would experience discomfort, the therapist operates a valve in a drain line that extends from the speculum whereupon the pressure decreases as waste water exits the colon. While drainage occurs the flow of fecal material and water is observed through a sight or view tube so that the therapist may observe the matter being discharged for diagnostic purposes. Once the pressure has dropped substantially, such as to one quarter pound per square inch, the drain valve is closes but the operative cycle repeats until the colon is well cleansed. However, in the event the attendant or therapist engages in conversation with the patient or becomes otherwise distracted, he or she may fail to observe that the pressure has increased beyond the upper limit. In this case the patient will normally experience discomfort or even pain. A potential for damage to the intestine will also arise.

To overcome this problem, U.S. Pat. No. 4,626,239 discloses a colonic lavaging machine comprising a housing, a water supply conduit mounted within said housing through which water may be fed from a water supply to a speculum, a water drain conduit mounted within said housing through which waste water may be drained from the speculum, means for sensing the pressure of water in said water supply conduit, and means coupled with said sensing means for controlling the flow of waste water in said water drain conduit responsive to a high and to a low level of water pressure sensed by said sensing means in said water supply conduit, said control means including an electrical pressure switch operatively associated with said water supply conduit, a solenoid valve operatively associated with said water drain conduit, and electrical conductor means coupling said solenoid valve with said pressure switch. However, the solenoid valve adapted on the drain conduit operates only when the pressure reaches above the maximum limit set by a therapist failure of which may lead to hazard and is based on electricity.

Accordingly, there is a need for a colon irrigation device and method which would prevent the potential for apparatus malfunction, reduce safety hazards on the apparatus, and provide more comfort by bringing lesser cramping pains to the patient.

Further problem is associated with the servicing of the device without removal of pipeline and electric circuit.

The present invention therefore is directed at alleviating these just described problems and limitations associated with colon irrigation machines of the prior art.

### SUMMARY OF THE INVENTION

In one embodiment, the present invention provides a colon irrigating device comprising a speculum for insertion into rectum of colon for irrigation and extracting matter, a water supply line connected with the said speculum, a drain line connected with the said speculum, and a bypass line connected to the water supply line. According to the present invention, the bypass line includes a non-return valve operating in proportional to a back pressure developed in the colon thereby allowing gentler filling of colon and limiting the pressure of water in the colon to a maximum safe pressure.

In another embodiment the present invention provides a colon irrigating device comprising a housing having a back panel, and a frame made of a top panel, a bottom panel, a front panel and opposing side panels wherein the frame is adapted hingedly on to the back panel to facilitate easy access to interior of device either by swing opening the frame of the housing or the back panel for servicing without disconnecting water and electrical supply.

The present invention also provides a method for performing colon irrigation comprising steps of attaching a colon irrigating device of the present invention to a source water, adjusting pressure of the source water to a predetermined set pressure, directing the pressure adjusted water to colon for irrigating same and extracting waste material therefrom, and causing the water and waste material of various consistencies to flow from the patient's colon to a viewing and discharge station via a waste hose. The non-return valve on the bypass line opens in proportion to the back pressure developed in the colon thereby allowing gentle raising of pressure in the colon and opens full at a predetermined maximum back pressure thereby controlling pressure within the colon.

Further advantage of the present invention will become apparent to those skilled in the art upon reading and understanding the following detailed description of the preferred embodiments.

### BRIEF DESCRIPTION OF THE DRAWINGS

The accompanying drawings, which are incorporated into and form a part of the specification, schematically illustrate one or more exemplary embodiments of the present invention and together with the general description given above and detailed description of the preferred embodiments given below serve to explain the principles of the invention:

FIG. 1 shows a perspective rear view of a colon irrigation device without a back panel according to the present invention;

FIG. 2 shows a typical connection between a water supply line, a drain line and a bypass line according to an embodiment of the present invention; and

FIG. 3 shows a perspective view of swing opened frame colon irrigation device according to the present invention.

### DETAILED DESCRIPTION OF THE PRESENT INVENTION

The present invention provides a highly effective device and method for performing human colon irrigation.

In general, the colon irrigation device of the present invention comprises a bypass line having a non-return valve connected to a warm water supply line wherein the non-return valve operates in proportional to a back pressure developed in the colon thereby allowing gentler filling of colon and limiting the pressure of water in the colon to a maximum safe pressure.

The present invention also provides a novel housing for a colon irrigation device to provide easy access of internal part of the colon irrigation device without disconnecting the water supply line and electric supply of the colon irrigation device.

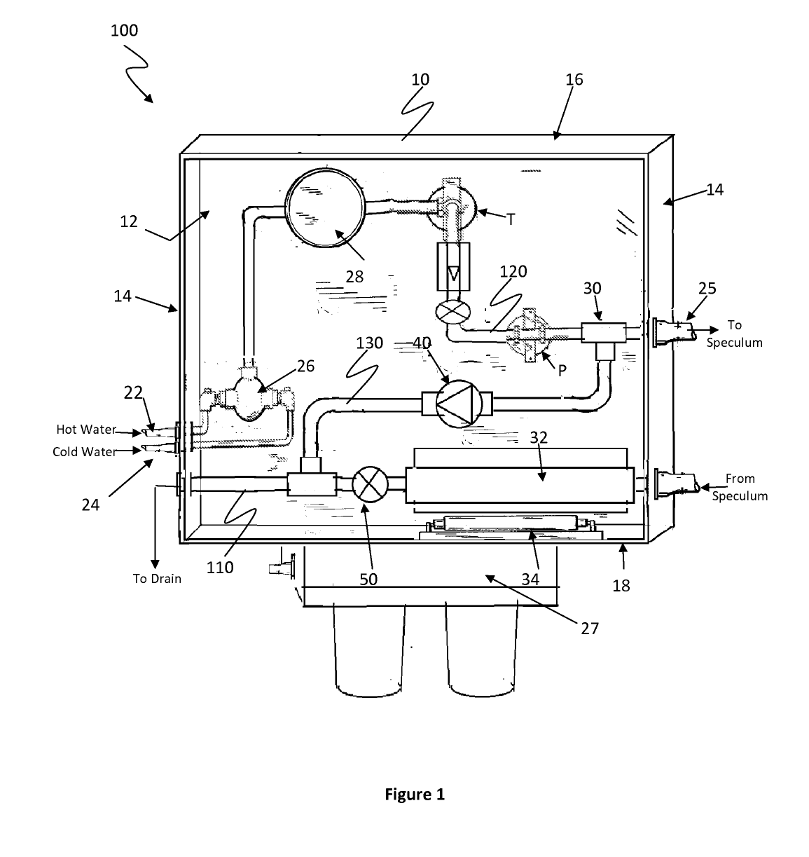

Referring to the FIGS. 1, 2 and 3, there is illustrated a colon irrigation device (100) incorporating the features according to the preferable embodiment of the present invention. The colon irrigating device (100) includes a housing (110), a water supply line (120), a drain line (130), a bypass line (140) and a speculum (150). The housing (110) comprises a frame (10) and a back panel (15). The frame is made of a front panel (12), opposing side panels (14), a top panel (16) and a bottom panel (18).

The water supply line (120) connects the source water to the speculum (150). The water supply line (120) includes a hot water conduit (22) and a cold water conduit (24) on one of the opposing side panels (14). The hot and cold water conduits (22, 24) are connected to a water mixing valve (26) for adjusting temperature of water. Water supply line (120) is connected with warm water through the filters. The warm water from the filters is directed through UV processing means (27), which is connected to the pressure control valve (28) to reduce pressure of warm water to a predetermined set pressure which can be anywhere between 1 and 5 psi according to the present invention. The filtering and UV process are very well known and not required to describe herein detail. The pressure control valve (28) is connected to an outlet conduit (25) through a tee (30). The pressure (P) and temperature (T) devices are connected on the water supply line (120) by known ways and are not required to describe in detail.

The bypass line (140) having a non-return valve (40), is connected to the supply line through tee (30). The non-return valve (40) according to the present invention opens under pressure and in proportion to the back pressure developed by the colon while filling the water in the colon which is referred hereinafter to as “back pressure developed in the colon”. The non-return valve (40) according to the present invention is a spring loaded valve. According to the present invention, the non-return valve (40) starts opening at a back pressure of about 0.3 psi and opens full at a maximum back pressure of about between 1 and 5 psi, according to the pre-determined maximum back pressure.

The water supply line (120) through the outlet conduit (25) extends from the housing (110) and connected to the speculum (150). The present invention uses a conventional speculum (150) and is not required to describe in detail. The speculum (150) is adapted for insertion into the anal canal of a patient. Thus, the warm water from the water supply line (120) passes through the speculum (150) into the colon of a patient where it will act to loosen feces matters by an irrigation method. As soon as water starts filling the colon, a back pressure starts developing in the supply line. When the back pressure reaches to 0.3 psi, the non-return valve (40) starts by-pass flow of the water by opening in proportion to the back pressure developed in the colon. When the pressure crosses the predetermined maximum back pressure, the non-return valve (40) opens full thereby limiting the pressure to a set pressure value in the colon. According to the present invention, the predetermined maximum back pressure is 2 psi. As the non-return valve opens in proportion to the back pressure developed in the colon, the pressure within the colon rises gently thereby allowing gentler filling of colon and full opening of the valve limits the pressure of water in the colon to a maximum safe pressure. In this way, the present invention helps to raise the pressure gently in the colon and safeguards from rising of the pressure above the maximum safe allowable pressure within the colon which makes fill comfort to the patient and safeguards from damaging of the intestine by not allowing the pressure in the colon to increase beyond the upper limit.

A disinfecting or cleaning solution can be mixed with warm water from the water mixing valve (26) for the purpose of disinfecting or cleaning the drain line (130) and/or all the internal water lines except those in the filtration system. This is done after and/or between treatments.

When the colon becomes full of water, a therapist commences a reverse flow by opening the drain valve (50) on the drain line. The reverse flow contains water and extracted material which flows through the speculum (150) into the drain line (130). The drain line (130) comprises an optically transparent tube (32) below which, by a known way, an illumination means (34) is arranged to view and observe the waste water and extracted material from colon.

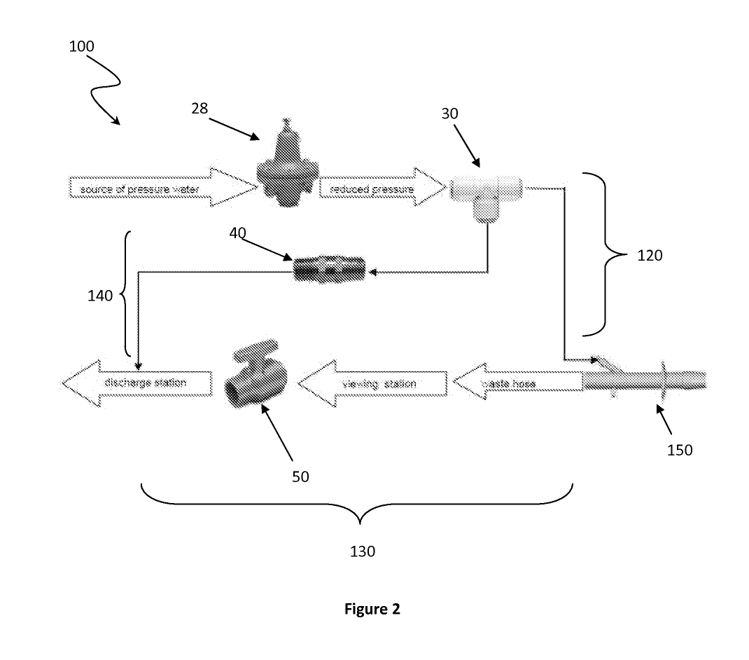

As shown in the FIGS. 1 and 2, outlet of non-return valve (40) on the bypass line (140) is connected to drain line (130). Alternatively, outlet of the bypass line can be connected to other than drain line.

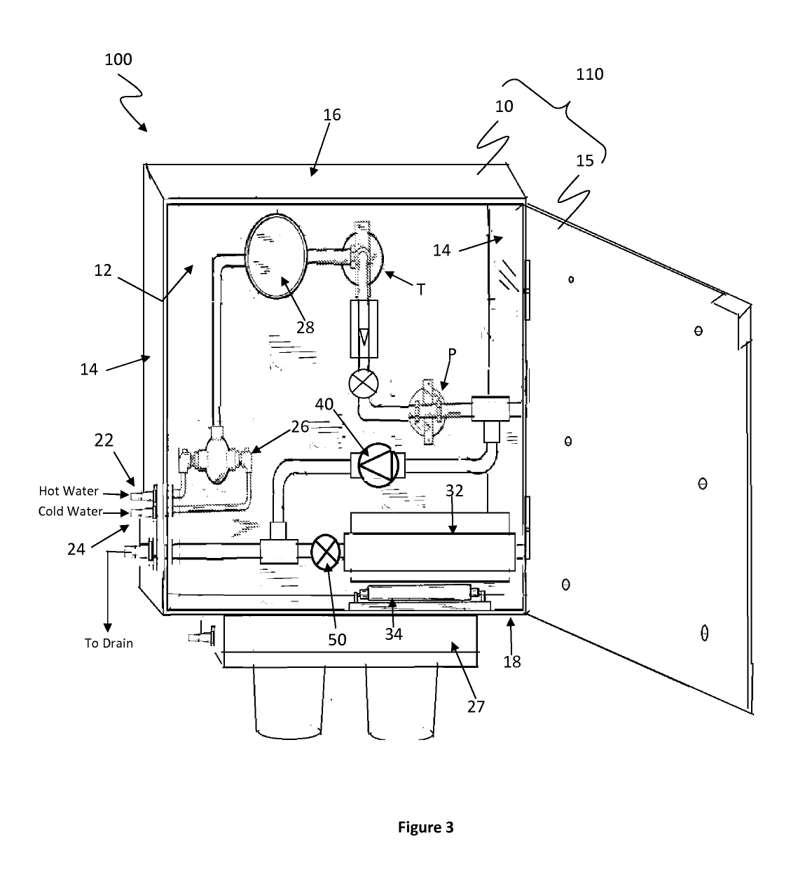

Referring FIG. 3 shows a perspective view of a swing opened colon irrigating device according to another embodiment of the present invention. As shown in the FIG. 3, the housing (110) comprises a back panel (15) is connected hingedly to the frame (10). The frame includes a front panel (12), opposing side panels (14), a top panel (16), and a bottom panel (18). The internal parts such as the supply line and the drain line along with equipments such as mixing valve, pressure control valve, pressure and temperature gauges, viewing glass, drain valve etc. are adapted over the front panel (12) of the frame (10) of the housing (110). This allows easy access to the interior of the device either by swing opening the frame (10) of the housing (110) or the back panel (15) for servicing without disconnecting water and electrical supply. This also allows servicing, calibrating and testing of the colon irrigating device while operating.

According to the present invention, the hinge is pin-hole type. The pins (not shown) are adapted over the back panel (15) or on the wall and holes (not shown) are adapted over the frame (10). Alternatively, the pins can be adapted fixedly over the wall which can act as a back panel of the device. In the closed position, the colon irrigating device is secured “closed” by means of a known fastener means such as a screw or a latch.

According to the present invention, the housing (110) can be mounted on a wall by fastening the back panel (15) on the wall by a known means such as screws, nails etc. When the back panel (15) is secured, the frame (10) can be opened by swinging away from the back panel (15) as and when required.

If the colon irrigation device is adapted on a platform such as a counter, stand or movable stand then, the back panel can be opened by swinging away from the frame (10) of the colon irrigation device.

The foregoing is illustrative of the present invention and is not to be construed as limiting thereof. The electric connection and other known parts are not shown and described herein in detail as the person skilled in the art can easily implement those things in the present invention, Further though a few exemplary embodiments of the present invention have been described, those skilled in the art will readily appreciate that many modifications are possible without materially departing from the novel teachings and advantages of the present invention. Accordingly, all such modifications are intended to be included within the scope of the present invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein:

What is claimed is:

1. A colon irrigation device, comprising
a speculum for insertion into rectum of colon for irrigation and extracting matter;
a water supply line connected with the said speculum;
a drain line connected with the said speculum; and
a bypass line connected to the water supply line, said bypass line includes a non-return valve operating in proportional to a back pressure developed in the colon thereby allowing gentler filling of colon and limiting the pressure of water in the colon to a maximum safe pressure.

2. The colon irrigation device of claim 1, wherein the non-return valve is a spring loaded valve.

3. The colon irrigation device according to claim 1, wherein the non-return valve opens at a back pressure of about 0.3 psi.

4. The colon irrigation device according to claim 1, wherein the non-return valve opens full at a predetermined maximum back pressure.

5. The colon irrigation device according to claim 4, wherein the predetermined maximum back pressure for full opening of the non-return valve is anywhere between 1 psi and 5 psi.

6. The colon irrigation device according to claim 1, wherein the non-return valve opens full at a back pressure anywhere between 1 psi and 5 psi.

7. The colon irrigation device according to claim 1, wherein outlet of the bypass line is preferably connected with the drain line of the colon irrigation device.

8. A colon irrigation device, comprising:
a housing having a back panel, and a frame made of a front panel, a top panel, a bottom panel and opposing side panels; and
wherein a water supply line through which water is fed to speculum from water supply, a drain line mounted though which waste water is drained from the speculum and a bypass line having a non-return valve operating under back pressure, connected to the water supply line are adapted in the said frame of the housing;
wherein said frame is adapted hingedly on to the back panel to facilitate easy access to interior of device either by swing opening the frame of the housing or the back panel for servicing without disconnecting water and electrical supply and while water is flowing through the device.

9. The colon irrigation device according to claim 8, wherein the housing can be mounted on a wall by fastening the back panel on the wall.

10. A method for performing colon irrigation, said method comprising steps of
i) attaching a colon irrigating device to a source of water, wherein said colon irrigation device further comprises of a speculum for insertion into rectum of colon for irrigation and extracting matter; a water supply line connected with the said speculum; a drain line connected with the said speculum; and a bypass line connected to the water supply line, said bypass line includes a non-return valve operating under a back pressure for allowing gentler filling of colon and limiting the back pressure of water in the colon to a maximum safe pressure;
ii) adjusting pressure of the source water to a predetermined set pressure;
iii) directing the pressure adjusted water to colon for irrigating same and extracting waste material therefrom; and
iv) causing the water and waste material of various consistencies to flow from the patient's colon to a viewing and discharge station via a waste hose;
wherein non-return valve on the bypass line opens in proportion to the back pressure developed in the colon thereby allowing gentle raising of pressure in the colon and opens full at a predetermined maximum back pressure thereby controlling pressure within the colon.

11. The method for performing colon irrigation according to claim 10, wherein the predetermined set pressure of source water is anywhere between 1 psi and 5 psi.

12. The method for performing colon irrigation according to claim 10, wherein the non-return valve starts opening at a back pressure of about 0.3 psi.

13. The method for performing colon irrigation according to claim 10, wherein the non-return valve opens full at a back pressure anywhere between 1 psi and 5 psi.

14. A colon irrigation device, comprising: a housing having a back panel and a frame made of a front panel, a top panel, a bottom panel and opposing side panels, a water supply line mounted within the frame of said housing through which water is fed to speculum from water supply and a water drain conduit mounted within the frame of said housing though which waste water is drained from the speculum wherein said frame is adapted hingedly on to the back panel to facilitate easy access to interior of device either by swing opening the frame of the housing or the back panel for servicing without disconnecting water supply and electrical supply.

15. A colon irrigation device according to claim 14, wherein the frame is adapted hingedly on to the back panel with a pin-hole type hinge.

16. A colon irrigation device according to claim 14 or 15, wherein a wall can act as a back panel of said housing of the colon irrigation device.
